(19) United States
(12) Patent Application Publication
(10) Pub. No.: US 2010/0198233 A1
(43) Pub. Date: Aug. 5, 2010

(54) EPILATOR HEAD FOR TRAPPING HAIR AND EPILATOR WITH SUCH HEAD

(76) Inventor: Yehuda Poran, Golan Heights (IL)

Correspondence Address: WESTMAN CHAMPLIN & KELLY, P.A., SUITE 1400, 900 SECOND AVENUE SOUTH, MINNEAPOLIS, MN, 55402, US

(21) Appl. No.: 12/669,231

(22) PCT Filed: Aug. 9, 2007
(86) PCT No.: PCT/IB2007/002957

§ 371 Date: Jan. 15, 2010

(30) Foreign Application Priority Data
Jul. 18, 2007 (EP) 07 01 4090.0

(51) Int. Cl. A61B 17/30

(52) U.S. Cl. 606/133

(57) ABSTRACT

The invention is directed to an epilator head for trapping hair for removal. The head comprising a rotating body which has an axis of rotation and is adapted to be rotated by a motor. At least one pair of pincers is arranged at or in the rotating body. The pair of pincers are able to rotate in accordance with the rotation of the rotating body. At least their outermost ends are adapted to move towards each other and away from each other depending on the rotary location of the rotating body. According to an aspect of the invention at least one intermediate member is provided which is arranged such that it is at least linearly moveable between the outermost ends of the pincers. Another aspect of the invention is that the pair of pincers only extend on one side of the axis of rotation. A further aspect of the present invention provides at least one spring which is located between the pair of pincers and which is arranged to bias the pincers either away from each other or towards each other. According to a further aspect of the present invention at least one actuator is provided for the pair of pincers. It is preferably supported to move along or parallel to the axis of rotation and engages a first pincer adjacent an end thereof being distal to the axis of rotation and a second pincer at or adjacent an end thereof being proximal to the axis of rotation.

### FIELD OF THE INVENTION

The present invention relates to a hair removal system, particularly to a epilator head for trapping hair and an epilator with such head.

### BACKGROUND OF THE INVENTION

Hair removing devices use one of two methods for removing hair. In one method, the hair is cut, leaving the roots intact beneath the skin surface. This is commonly named depilation. In the other method, hair is removed by pulling it out from its roots. This is commonly called epilation.

There are several mechanisms for removing hair according to the latter method. Disk mechanisms are often used. In general, these mechanisms include discs and associated pincer-like elements. When two pincer-like elements are brought close together, hair is trapped between them. The discs, which rotate and produce a torque, then uproot the hair trapped between their associated pincers. The pincer-like elements and their associated discs move in unison and all pincer-like elements within a fixed distance move close to their adjacent pincer-like elements synchronously. The forces required in such mechanisms are multiples of the number of pincers. In some of these epilatory devices, the disc mechanisms have cylindrical shapes.

Other epilating devices which use disc mechanisms employ a large spring with bearings connected to its ends. In such devices, the spring presses on the bearings producing a constant force which acts identically over all the disks and their associated pincer-like elements. The magnitude of the force is the same throughout the entire mechanism. The forces required in such mechanisms are relatively small and the energy required is not great.

Several of the proposed disk hair removal systems involve the use of tilted discs which come together at a point to grasp one or more strands of hair. Other disc mechanisms involve the use of cams to alternately bring the discs together and apart, thereby trapping strands of hair. Various disc mechanisms are discussed in U.S. Pat. Nos. 4,935,024; 5,057,115; 5,190,559; 5,797, 925; 5,857,903; 5,312,419; 5,196,021; 5,281,233; and 5,462,557.

U.S. Pat. No. 5,190,559 is directed to an epilating appliance with a casing for accommodating a rotary cylinder mounted on an axle and adapted to be driven by a motor. The rotary cylinder has associated to it a plurality of pinching members provided with cutouts and extending from one side of the rotary cylinder to the opposite side. The pinching members are capable of pivoting and moving into contact with each other in pairs by means of actuating members located at a distance to the axle of the rotary cylinder and displaceable against the action of at least one spring. In order to increase the lever arm for pivotal movement of the pinching members, the pivot axis of the pinching members of the rotary cylinder extends at a large relative distance to the axis of the rotary cylinder.

A epilating device is subject to several constraints. The pincer-like elements associated with each disk must close quickly. The pressure exerted by each contacting pair of pincers must be neither too great nor too little. In the former case, the hair would be cut, while in the latter case, the hair would slide through without being pulled out at its roots. Typically, all the pincers associated with a row of disks must contact their adjacent pincers simultaneously. Lastly, the contacting mechanism must be simple, operate reliably over time, and be easy to maintain.

Presently, there is a need for a epilating device that is easy and inexpensive to assemble and to maintain and which can uproot a greater number of hairs over a larger area than is possible using prior art devices. In addition, there is also an ongoing need for a epilating device that reduces discomfort associated with hair removal.

### SUMMARY OF THE INVENTION

It is an object of the present invention to provide an improved epilator head, epilator with such head and an improved epilating method.

This object is attained with the subject-matter according to the claims.

The invention is directed to an epilator head for trapping hair for removal. The term epilating in this context does not mean that all hears are plugged out with their roots. It just means that the hairs are gripped and pulled from the skin. Some roots may remain in the skin. The head according to the invention comprising a rotating body which has an axis of rotation and is adapted to be rotated by a motor. Preferably the rotating body is a drum-like or cylindrical body rotating around the axis of rotation. At least one pair of pincers is arranged at or in the rotating body which are essentially radially arranged to the axis of rotation. The pair of pincers are able to rotate in accordance with the rotation of the rotating body. At least their outermost ends are adapted to move towards each other and away from each other depending on the rotary location of the rotating body. This is an arrangement which is essentially common for all aspects of the present invention.

According to an aspect of the invention at least one intermediate member is provided which is arranged such that it is at least linearly moveable between the outermost ends of the pincers. This is preferably attained with a floating, sliding or free bearing of the intermediate member.

The invention therefore has the preferred advantage that the pincers are moving simultaneously in opposite directions until they abut the intermediated member. The result of the floating intermediate member is equal forces between the edges or outermost ends of the pincers and the intermediate member. Preferably two pairs of pincers are arranged on a common supporting member and the same mirrored mechanism is provided at the other side. Then the force of all four pincers is equal. The total force required is P×2.

In U.S. Pat. No. 5,190,559 tow bars move in opposite directions. Each bar pushes a set of pinches. When the first ones touch each other, the other pincers close by applying force to close them in an elastic way. The total force is bigger than P×N. The total force required in this case is bigger than 9×P.

It is thus a preferred advantage of the mechanism according to the invention to get equal forces between the pincers and to apply a much smaller total force with a much bigger production tolerance. Moreover, in the above cited prior art all of the pincers cannot close. According to the present invention the intermediate member moves a little and the force between it and the pincers will by equal.

Another aspect of the invention is that the pair of pincers only extend on one side of the axis of rotation. This enables an individual actuation of the active or outermost ends of the pincers and relatively small space for each pair of pincers.

A further aspect of the present invention provides at least one spring which is located between the pair of pincers and which is arranged to bias the pincers either away from each other or towards each other. Preferably, the pincers are biased away from each other. This particularly assists the quick opening of the pincers without any further action or mechanics being required. Particularly with the floating intermediate member holding the spring between the pincers this arrangement is small, effective and able to also apply equal opening forces to the pincers applied by the spring.

According to a further aspect of the present invention at least one actuator is provided for the pair of pincers. It is preferably supported to move along or parallel to the axis of rotation and engages a first pincer adjacent an end thereof being distal to the axis of rotation and a second pincer at or adjacent an end thereof being proximal to the axis of rotation. This aspect alone and particularly together with any of the other aspects described above provides a simple but effective mechanism for actuating the pincers, particularly with a relatively small force and resulting equal pincing forces.

As mentioned above, the intermediate member preferably holds the spring in place, the spring being preferably a cylindrical spring which biases the pair of pincers preferably away from each other, and wherein the intermediate member is floatingly supported between the or each pair of pincers.

Further preferably a supporting member is provided which supports at least the pair of pincers and freely, floatingly or slidingly supports the intermediate member and the actuator, preferably along a common or parallel axis of movement. The intermediate member is preferably able to be clicked into the supporting member. The supporting member is preferably a generally rail-shaped member preferably made of metal and additionally supports a second pair of pincers and a respective intermediate member and actuator. This results in preferably two pincing mechanisms on one supporting member comprising each a pair of pincers, an intermediate member, a spring and an actuator. The pincers and the intermediate member are preferably formed to be able to be clicked into the rail-shaped supporting member.

The intermediate member preferably has radial protrusions distal to the rotating axis at its longitudinal ends so as to form a clamping arrangement with the respective abutting pincers. These protrusions have a shape generally corresponding to the respective pincer abutting.

Preferably a cam-like means is provided at or adjacent to the rotating body, wherein the actuator is arranged to be pushed by the cam-like means and thereby to make the pincers to move towards each other and preferably towards the intermediate member. The actuator is also arranged to be biased against this cam means by the spring and via the pincers.

The head preferably comprises head gear means for transferring a movement from the motor to the rotating body so as to rotate the rotating body. These head gear means are preferably provided on one side of the rotating body of the head.

The cam means further preferably comprises a pressing wheel fixed in position and a rotating revolving means for slidingly holding the actuator. The actuator's outer end preferably protrudes out of revolving means and can be pushed inwardly through the revolving means by the pressing wheel when the actuator's outer end comes into engagement therewith. Other cam means may also be suitable or any other (cam like) equivalent element that would perform the same or similar action or effect.

Preferably one of the pair of revolving means has outer circumferential gear teeth for engagement with a transmission gear and for transferring a rotational movement from the motor.

Preferably, the head can be decoupled from an epilator body. In the affirmative it comprises a coupling means, preferably a removable pair of latches, for coupling it to the epilator body.

The epilator head according to the present invention also embraces a cover with a window for just leaving open the pincers and related elements to trap and remove body hair. The cover is preferably made of plastic material and can be clicked onto the epilator head.

The present invention also concerns an epilator with the above described epilator head and an epilator body with a motor and preferably gear means. The epilator head can be removable and interchangeably attachable to the epilator body.

In another aspect the present invention is also directed to a method for epilating hair for its removal, particularly with an epilator head or an epilator according to any one of the preceding embodiments and aspects of the invention. The method comprising the steps of rotating a rotating body which has an axis of rotation, providing at least on pair of pincers, arranging the pair of pincers essentially radially to the axis of rotation with at least one intermediate member at least between outermost ends of the pair of pincers, and rotating the pincers in accordance with the rotation of the rotating body, and controlling the movement of the pincers so that at least their outermost ends move towards each other and to the intermediate member and away from each other depending on the rotary location of the rotating body. These steps are common for the following different method aspects of the invention.

According to one aspect the intermediate member is arranged so that it is at least linearly moveably between the outermost ends of the pincers. According to another aspect of the invention there is additionally the step of providing at least one pair of pincers only on one side of the axis of rotation. Another aspect is directed to the step of providing at least one spring between the pair of pincers so as to bias the pincers either away from each other or towards each other. A still further aspect of the invention concerns the step of providing at least one actuator for the pair of pincers and moving it along or parallel to the axis of rotation and engaging a first pincer adjacent an end thereof being distal to the axis of rotation and a second pincer at or adjacent an end thereof being proximal to the axis of rotation.

### BRIEF DESCRIPTION OF THE DRAWING

For a better understanding of the invention the following figures show or exemplify preferred embodiments.

FIG. 1 shows an exploded perspective view of an epilator head, a cover and an epilator body.

FIG. 2 shows a perspective view of an epilator head attached to driving means contained within an epilator body (not shown).

FIG. 3 shows a similar perspective as FIG. 2 but with the depilator head being removed from the driving means of the epilator body and being disassembled in part.

FIG. 4 shows a perspective and in part exploded view of the pincing components and related parts.

FIG. 5 shows a cross-section through the epilator head along the axis of rotation.

FIG. 6 shows the pincing components in assembled interaction in an open and a closed status.

FIG. 7 shows a schematic drawing of the pincing components and related parts highlighting particularly the bearings and kinematics in the open (FIG. 7A) and in the closed (FIG. 7B) configuration.

FIG. 8 shows a schematic drawing of the two pairs of pincing components and related parts highlighting particularly the bearings and kinematics in the open (FIG. 8A) and the close (FIG. 8B) status.

### DETAILED DESCRIPTION OF THE INVENTION

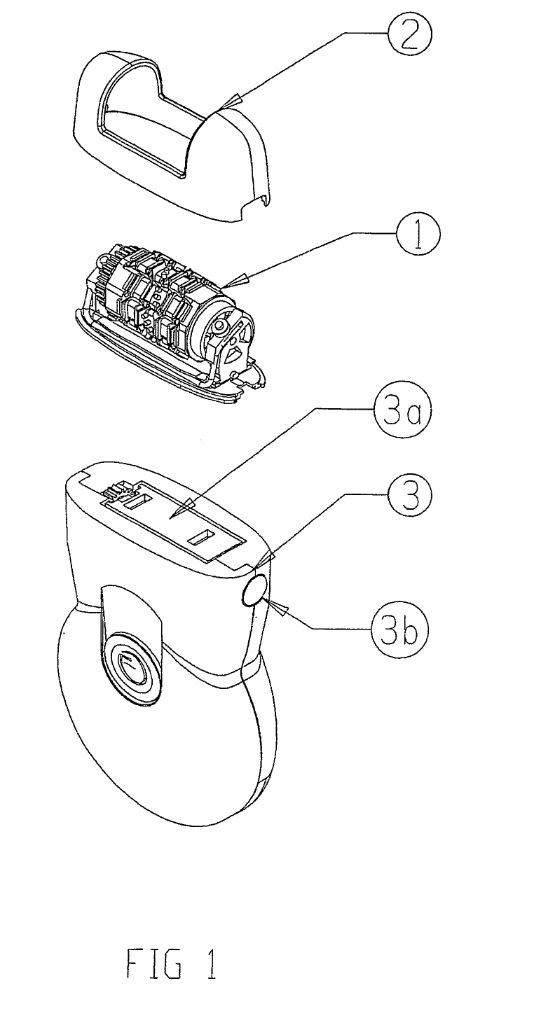

FIG. 1 displays an epilator head 1 according to a preferred embodiment of the invention, a lid or cover 2 therefor as well as an epilator body 3. The epilator head 1 is preferably able to be clicked onto the epilator body 3 and onto the respective adapting face 3a. A push knob 3b is preferably present in order to unlatch the epilator head 1 upon pressing the knob 3b. Any other known coupling and decoupling means can also be used.

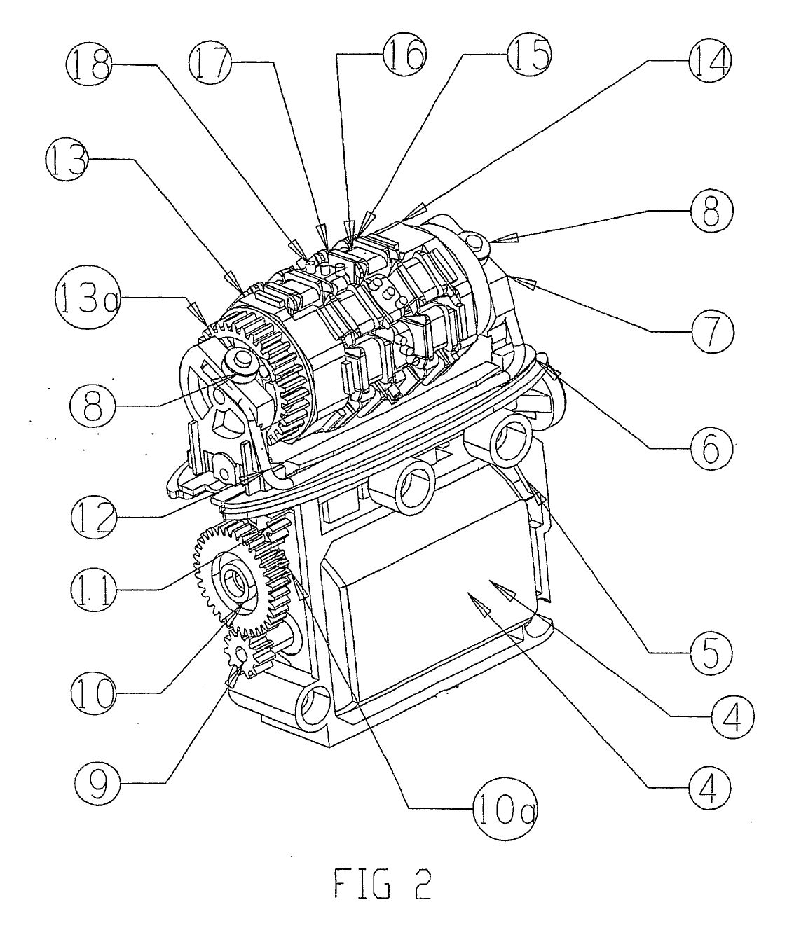

From FIG. 2 details of the epilator head and its coupling to the driving means within the epilator body (not shown) are apparent. In particular a motor 4 can be arranged in the epilator body by particular attachments 5. The attachments 5 can further comprise screws, snap-in latches or anything known in the art. A battery chamber or compartment (not shown) can be provided or a socket for a cable in order to supply energy to the motor 4.

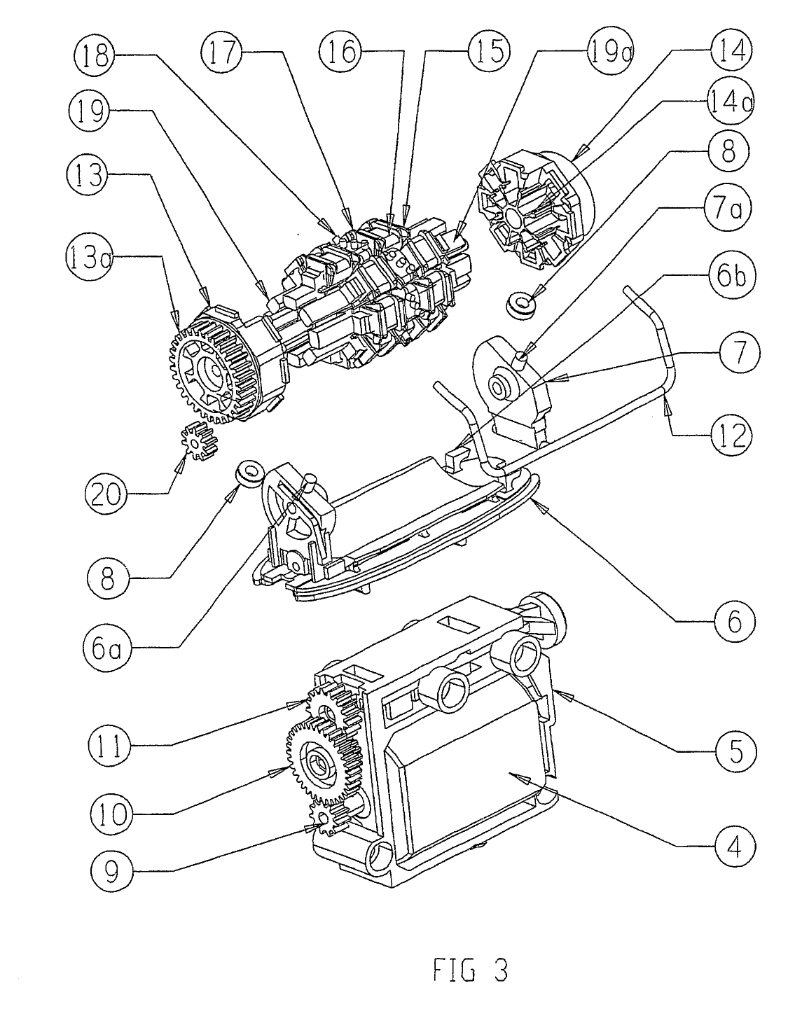

Latches 6 are shown forming the corresponding element to the adapting face 3a. A support 7 is also shown in FIG. 2 for rotationally holding a rotating body and a cam-like element, such as on either side a pressing wheel 8 and a revolver 13, 14 . The pressing wheel 8 is also rotationally held in place by a pin 7a of support 7 (shown better in FIG. 3). A spring 12 is provided to hold all elements of the support 7 in place and will be described in more detail below.

A motor gear wheel 9 is driven by the motor 4 and drives a first intermediate wheel 10 for a first preferred gear reduction. The first intermediate wheel 10 drives a connecting wheel 11 by another smaller second intermediate gear wheel 10a arranged on the same axle as the first intermediate gear wheel 10. This is a preferred arrangement of gear box within the epilator body.

In the epilator head preferably a first revolver 13 and a second revolver 14 are arranged for holding linearly movable actuators 19 in place. Both revolvers 13, 14 cooperate with the respective pressing wheel 8 to form a cam-like structure, as will be discussed below in more detail. Preferably one of the revolvers 13, 14, in the present case the first revolver 13, has gear teeth 13a for being driven by a gear wheel (shown with 20 in FIG. 3). By such preferred transfer of rotational movement the rotating body is rotated. Any other kind of rotational transfer is also covered by the present invention, including a translating movement provided by the rotating body and the conversion into a rotational movement.

FIG. 2 shows pincers 15, 17 and intermediate members 16 which will also be described in more detail below. In a preferred embodiment each pair of pincers 15, 17 is slightly offset to each pair neighbouring pair along the circumference of the rotating body. This assists that all hairs from a surface of user's skin can be removed.

A preferred intermediate support 28 is also already apparent which serves to support a holding rail for the pincing components and other related elements. On its outer circumference the intermediate support has preferably little pins for guiding the hair into the open pincers 15, 17 and the respective intermediate member 16.

FIG. 3 shows the separated motor and driving means from within the epilator body (not shown). Particularly the gear box and the connecting wheel 11 outputting a rotational force is better visible in FIG. 3. Alternatively, other known force outputting elements can be used such as a device outputting a reciprocating translational force.

In the upper portion of FIG. 3 the epilator head is shown with some parts being disassembled or being in the state before the complete assembly. The gear wheel 20 obtaining the rotational force from connecting wheel 11 when the epilator head is attached to the epilator body is apparent which drives the gear teeth 13a of the first revolver 13.

The support 7 is also derivable from this figure, particularly that it is preferably assembled from different elements which can be assembled by respective connectors 7b. The spring 12 preferably holds the individual elements of support 7 in place and is preferably made of an elastic material such as of spring metal. The spring 12 which is preferably generally U-shaped can be clipped into accordingly shaped grooves provided in the elements to be connected, preferably in the manner as shown best in FIGS. 2 and 3.

The revolvers 13, 14 are also shown in a disassembled state. Particularly in the revolver 14 it is apparent that recesses 14a are provided for ends 19a of the actuators 19. In the assembled state, these ends 19a of the actuators 19 extend through the revolvers 13, 14 at least in case the pincers 15, 17 are open. When such an extending end 19a of an actuator 19 passes the pressing wheel 8 it will be pressed into the rotating body so that the actuator 19 is linearly moved and can actuate the pincers 15, 17 in the manner described below in more detail.

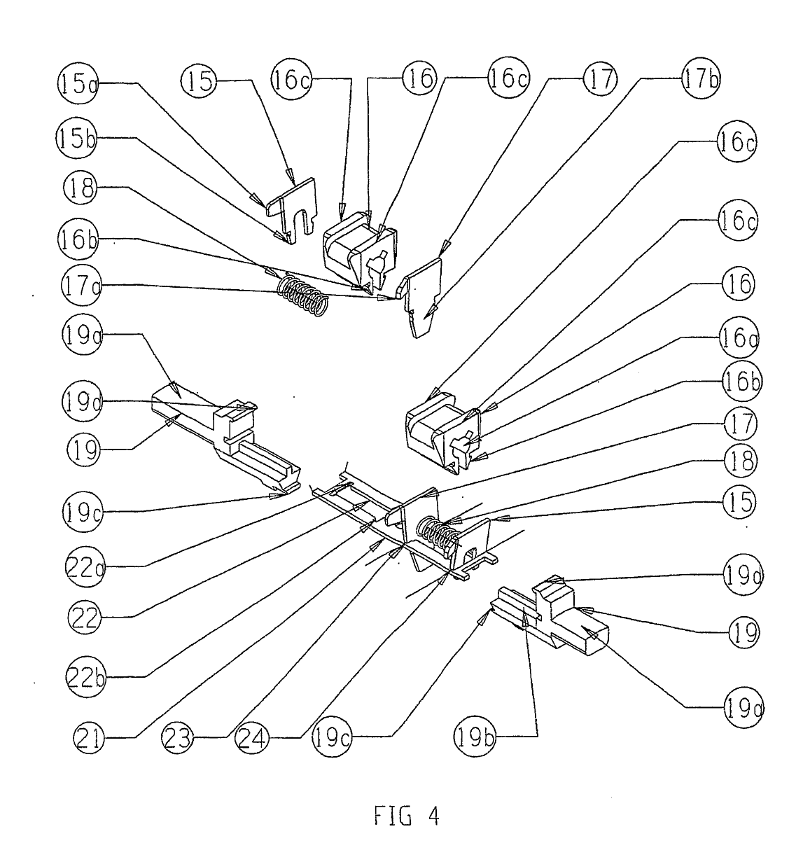

FIG. 4 shows particularly preferred pincing components and their related elements for driving them. The pincing components comprise the pincers 15, 17, the intermediate member 16 and a spring 8 which is preferably hold in place by a respective through going opening 16a in the intermediate member 16. The opening 16a is preferably a cylindrical bore for the preferred cylindrical spring 18. FIG. 4 shows for a better understanding the spring 18 in a position not being held by the intermediate member 16.

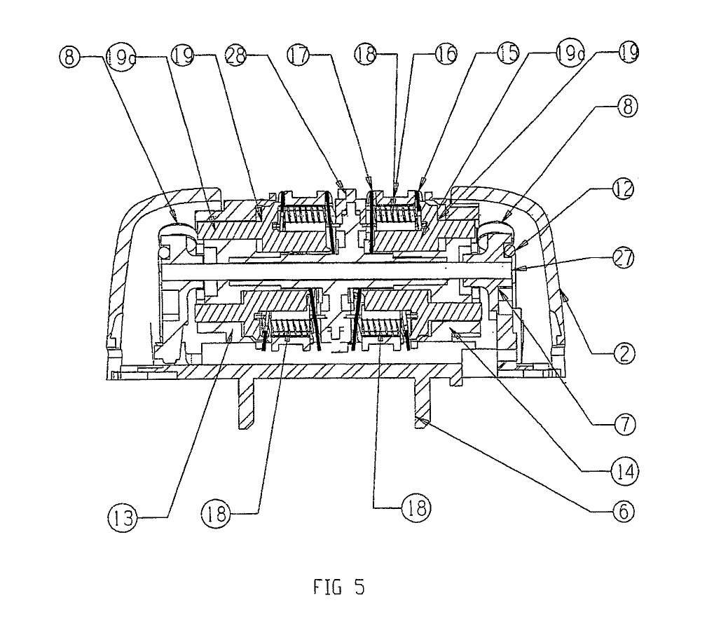

A supporting member 21 is shown which is preferably a rail-shaped element and made of an elastic material. Preferably it is made of spring metal so that element can be easily clicked into it and these elements can then be retained in the supporting member 21. Preferably the pincers 15, 17 have lower ends 15b, 17b which are shaped and adapted to be inserted into respective openings 22a, 22b, respectively. The intermediate member 16 also has a lower end 16b which is preferably elastically deformable due to its shape and can be inserted into a window 22 of the supporting member 21. The lower end 16b has preferably an arrow-shaped form in order to be able to stay retained in the supporting member 21 and to be linearly movable in its window 22. This can also be called a free, sliding or floating support or bearing. The intermediate member 16 preferably has radial ridges 16c which correspond in their design or form to the outermost ends of the pincers 15, 17 and which are intended to abut them or to trap hair between them and the respective pincer 15, 17. The supporting member 21 is preferably supported by another intermediate support (not shown in FIG. 4 but shown with reference numeral 28 in FIGS. 2 and 5).

The rotating body preferably comprises the revolvers 13, 14, the actuators 19, the pincers 15, 17, the intermediate members 16, the springs 18, the supporting members 21, and the intermediate supports. A preferred embodiment of the present invention comprises eight supporting members 21, each with two pairs of pincers 15, 17 (=32 pincers), 2 intermediate members 16 (=16 intermediate members) and on either of the two ends one actuator 19 (=16 actuators).

The actuator 19 is generally shaped like a half of a vice and preferably has a ridge 19b which can enter the open lower end 16b of the intermediate member 16 once the intermediate member 16 has been inserted into the window 22. This assists to bear the actuator 19 so that it can be moved linearly.

The actuator 19 further has a lower front face 19c for contacting and pushing the lower end 17b of the second pincer 17 when assembled. This lower end 17b can also be called proximal to the axis of rotation of the rotating member. Moreover, the actuator has an upper face 19d for contacting the first pincer 15 at a position adjacent its outermost end or adjacent its end which can also be called distal to the axis of rotation of the rotating member. With such an arrangement any force of the actuator 19 is equally distributed between the pincers 15, 17 and the linearly moveable intermediate member 16.

Pincers 15, 17 preferably can also comprise opening fingers 15a, 17a which are directed into the direction of rotation and which assist hair to be introduced into the pincers 15, 17 and the intermediate member 16. The ridges 16c at the intermediate member 16 also have preferred corresponding protrusions extending over the main body of the intermediate member 16 and being inclined to guide the hair in the afore described manner.

FIG. 5 particularly assists to show the interaction of the elements described before. First of all the lid or cover 2 is shown in a position preferably clicked onto the epilator head by means of latches 2a in a generally known manner. An upper window in cover 2 leaves particularly open the pincing components over a circumferential distance to allow the pincers 15, 17 and the intermediate member 16 to enter the window in an opened status, to then trap the hair, to close and to plug the hair from or out of the skin of a user in a generally known manner.

The preferred latches 6 are shown for removably attaching the epilator head to the epilator body (not shown) in a generally known manner.

An axle 27 is preferably provided to rotationally bear the revolvers 13 and 14 as well as the rotating body of the epilator head. The revolvers 13, 14 are rotationally connected or coupled to the rotating body so that a rotational movement of the revolvers caused by the driving means results in a rotational movement of the rotating body. The revolvers 13, 14 and further elements of the epilator head are held in place by the spring 12.

It is further derivable from FIG. 5 that the pressing wheel 8 engages an actuator's end 19a in case it arrives at the position of the pressing wheel 8. This causes the actuator to move linearly inwardly and the make each pair of pincers 15, 17 to move against each other or against the intermediate member 16 (see the upper part of FIG. 5). In the lower part of FIG. 5 it is shown how the spring 18 being held in place by the intermediate member 16 biases the pincers 15, 17 outwardly which causes the actuator 19 to move outwardly if not being engaged by the pressing wheel 8.

The intermediate support 28 is also shown with one of preferably three little guiding pins being intersected.

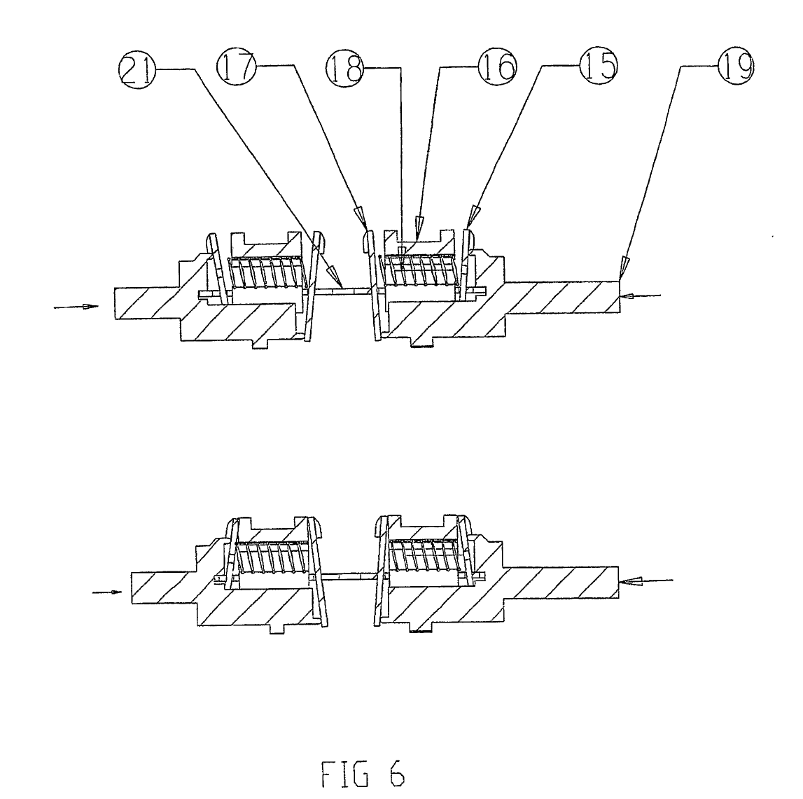

FIG. 6 shows the interaction of the pincers 15, 17, the intermediate member 16 as well as the actuators 19 with the preferred arrangement of two pairs of pincers 15, 17 on one supporting member 21. The upper part of FIG. 6 shows the pincers 15, 17 being in an open state, i.e. the outermost ends thereof being in an inclined position away from each other or the respective intermediate member 16. The lower part of FIG. 6 shows the same in a closed position and it is derivable from this figure that the actuators 19 have been moved linearly inwardly in order to close the pincers 15, 17 (preferably by the pressing wheel not shown). The engagement of each actuator 19 with the each pincer 15, 17 is also apparent. The first pincer 15 is shorter than the second pincer 17 and the first pincer 15 is engaged adjacent its outermost end which is distally located from the axis of rotation (not shown) and the second pincer 17 is engaged adjacent its innermost end which is proximally located from the axis of rotation.

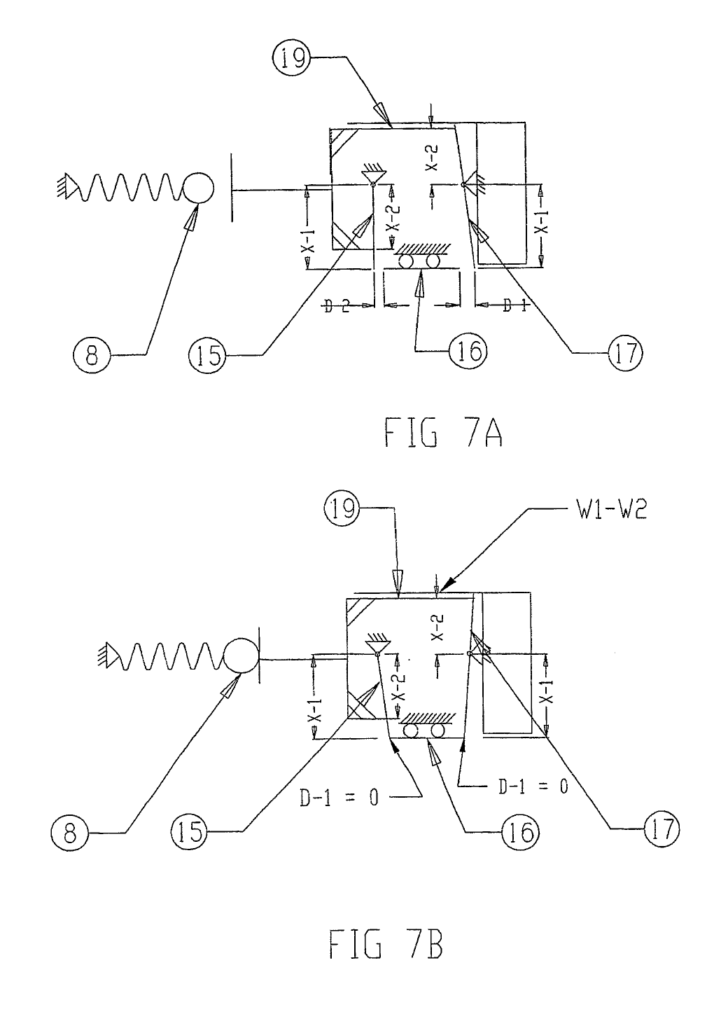

FIGS. 7A and 7B visualize how a preferred bearing of the different elements are realized and how the kinematics of the pincing components work. In FIG. 7A the floating bearing of the intermediate member is shown. In the preferred embodiment described above the supporting member (reference numeral 21 in FIG. 4) constitutes the basis of the bearing and a window (reference numeral 22 in FIG. 4) having a greater length than the engaged lower end (reference numeral 16b in FIG. 4) of the intermediate member 16 constitutes the guiding means. The structure of the supporting member and the pincers and their interaction constitutes the bearing for inclination or tilting for the pincers. It is also shown that the lever arms for the actuation of the pincers by the actuators is the same, namely X-2, and the length between the outermost ends of the pincers and the bearing is X-1, as shown in FIGS. 7A+B. i.e. the latter lengths being greater than the lever arms. The relatively large spring with a circle at its end on the left hand side of FIGS. 7A+B represent the pressure wheel. In FIG. 7A it is not in engagement while in FIG. 7B it is in engagement and drives the pincers and the intermediate member to the closed position or state.

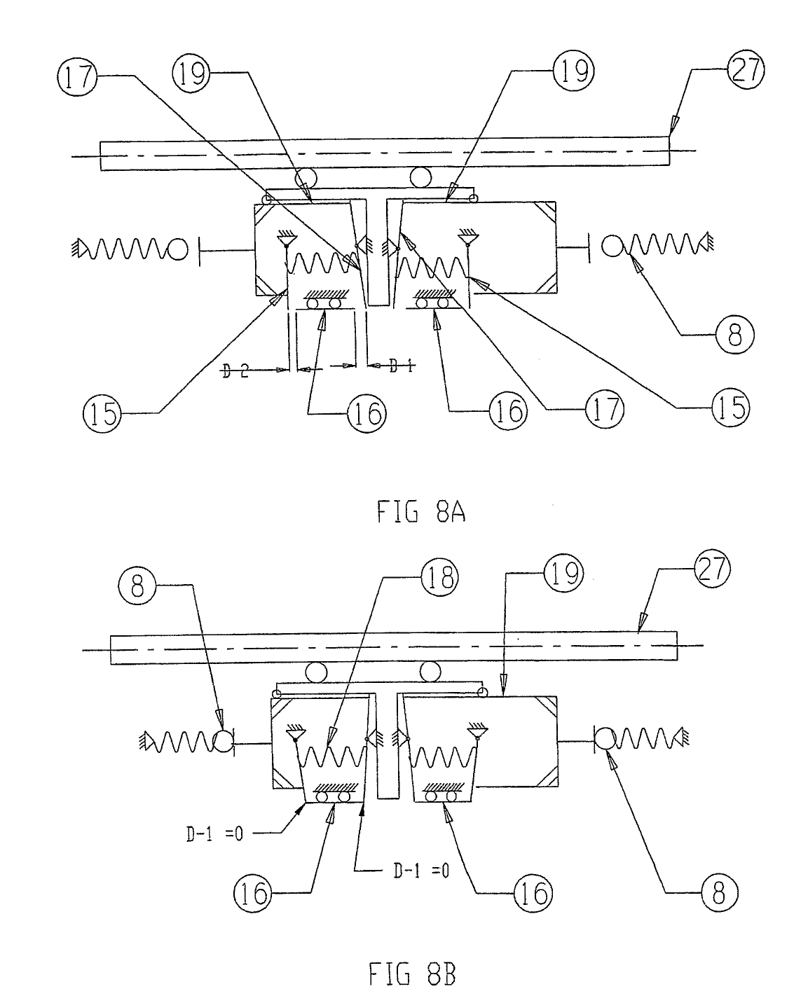

FIGS. 8A+B show the rotational bearing of the pincing components, i.e. the pincers 15, 17, the intermediate member 16 and the springs 18, on the axle 27. FIG. 8A shows the pincing components in an open state and FIG. 8B in a closed state. The nature of the bearings shown correspond to the ones depicted in FIGS. 7A+B.

The invention also covers all further features shown in the figures individually although they may not have been described in the afore description.

The present invention covers further embodiments with any combination of features from different embodiments described above.

The present invention also covers the exact terms, features, values and ranges etc. in case these terms, features, values and ranges etc. are used in conjunction with terms such as about, around, generally, substantially, essentially, at least etc. (i.e., “about 3” shall also cover exactly 3 or “essentially radial” shall also cover exactly radial).

1. Epilator head for trapping hair for removal, the head comprising: a rotating body which has an axis of rotation and is adapted to be rotated by a motor; and at least one pair of pincers, the pair of pincers being essentially radially arranged to the axis of rotation and being able to rotate in accordance with the rotation of the rotating body, and at least their outermost ends being adapted to move towards each other and away from each other depending on the rotary location of the rotating body; and at least one intermediate member which is at least linearly moveably arranged between the outermost ends of the pincers.

2. Epilator head for trapping hair for removal, the head comprising: a rotating body which has an axis of rotation and is adapted to be rotated by a motor; and at least one pair of pincers, the pair of pincers being essentially radially arranged to the axis of rotation and being able to rotate in accordance with the rotation of the rotating body, and at least their outermost ends being adapted to move towards each other and away from each other depending on the rotary location of the rotating body; wherein the pair of pincers only extend on one side of the axis of rotation.

3. Epilator head for trapping hair for removal, the head comprising: a rotating body which has an axis of rotation and is adapted to be rotated by a motor; and at least one pair of pincers, the pair of pincers being essentially radially arranged to the axis of rotation and being able to rotate in accordance with the rotation of the rotating body, and at least their outermost ends being adapted to move towards each other and away from each other depending on the rotary location of the rotating body; wherein at least one spring is located between the pair of pincers which is arranged to bias the pincers either away from each other or towards each other.

4. Epilator head for trapping hair for removal, the head comprising: a rotating body which has an axis of rotation and is adapted to be rotated by a motor; and at least one pair of pincers, the pair of pincers being essentially radially arranged to the axis of rotation and being able to rotate in accordance with the rotation of the rotating body, and at least their outermost ends being adapted to move towards each other and away from each other depending on the rotary location of the rotating body; wherein at least one actuator is provided for the pair of pincers which is supported to move along or parallel to the axis of rotation and engages a first pincer adjacent an end thereof being distal to the axis of rotation and a second pincer at or adjacent an end thereof being proximal to the axis of rotation.

5. Epilator head according to claim 3.

6. Epilator head according to claim 1, wherein the intermediate member holds the spring in place, the spring being cylindrical spring which biases the pair of pincers away from each other, and wherein the intermediate member is floatingly supported between the pair of pincers.

7. Epilator head according to claim 5, wherein a supporting member is provided which supports at least the pair of pincers and slidingly supports the intermediate member.

8. Epilator head according to claim 7, wherein the supporting member, which is preferably a rail-shaped member preferably made of metal, additionally supports a second pair of pincers and a respective intermediate member and actuator.

9. Epilator head according to claim 2, wherein the intermediate member has radial protrusions distal to the rotating axis at its longitudinal ends so as to form a clamping arrangement with the respective pincers.

10. Epilator head according to claim 4, wherein the lever arm obtained by the engagement of each actuator with the first pincer and with the second pincer is essentially the same.

11. Epilator head according to claim 5, comprising a cam means provided at the rotating body, wherein the or each actuator is arranged to be pushed by the cam means and thereby to make the pincers to move towards each other and preferably towards the intermediate member, and wherein the actuator is also arranged to be biased against this cam means by the spring.

12. Epilator head according to claim 11, wherein the head comprises head gear means for transferring a movement from the motor to the rotating body so as to rotate it.

13. Epilator head according to claim 11, wherein the cam means comprises a pressing wheel fixed in position and a rotating revolving means for slidably holding the actuator, wherein an actuator's outer end protrudes out of the revolving means and can be pushed inwardly through the revolving means by the pressing wheel when the actuator's outer end comes into engagement therewith.

14. Epilator head according to claim 13, wherein one of preferably a pair of revolving means has outer circumferential gear teeth for engagement with a transmission gear and for transferring a rotational movement from the motor.

15. Epilator head according to any of the preceding claims, further comprising a coupling means for coupling it to epilator body, the coupling means preferably being a removable pair of latches.

16. Epilator with an epilator head according to any of the preceding claims and preferably a cover, and an epilator body with a motor, preferably a battery compartment and further preferably gear means.

17. Epilator according to claim 16, wherein the epilator head and cover is removably and interchangeably attachable to the epilator body.

18. Method for epilating hair for its removal, particularly with an epilator head or an epilator, the method comprising the steps of: rotating a rotating body which has an axis of rotation; providing at least on pair of pincers, arranging the pair of pincers essentially radially to the axis of rotation with at least one intermediate member at least between outermost ends of the pair of pincers, arranging the intermediate member so that it is at least linearly moveably between the outermost ends of the pincers and rotating the pincers in accordance with the rotation of the rotating body, and controlling the movement of the pincers so that at least their outermost ends move towards each other and to the intermediate member and away from each other depending on the rotary location of the rotating body.

19. Method for epilating hair for its removal, particularly with an epilator head or an epilator according to any one of the preceding respective claims, the method comprising the steps of: rotating a rotating body which has an axis of rotation; providing at least one pair of pincers only on one side of the axis of rotation, arranging the pair of pincers essentially radially to the axis of rotation, and rotating the pincers in accordance with the rotation of the rotating body, and controlling the movement of the pincers so that at least their outermost ends move towards each other and away from each other depending on the rotary location of the rotating body.

20. Method for epilating hair for its removal, particularly with an epilator head or an epilator, the method comprising the steps of: rotating a rotating body which has an axis of rotation; providing at least one pair of pincers, arranging the pair of pincers essentially radially to the axis of rotation with at least one spring between the pair of pincers so as to bias the pincers either away from each other or towards each other, and rotating the pincers in accordance with the rotation of the rotating body, and controlling the movement of the pincers so that at least their outermost ends move towards each other and away from each other depending on the rotary location of the rotating body.

21. Method for epilating hair for its removal, particularly with a an epilator head or an epilator, the method comprising the steps of: rotating a rotating body which has an axis of rotation; providing at least on pair of pincers, arranging the pair of pincers essentially radially to the axis of rotation, and rotating the pincers in accordance with the rotation of the rotating body, and controlling the movement of the pincers so that at least their outermost ends move towards each other and away from each other depending on the rotary location of the rotating body, and providing at least one actuator for the pair of pincers and moving it along or parallel to the axis of rotation and engaging a first pincer adjacent an end thereof being distal to the axis of rotation and a second pincer at or adjacent an end thereof being proximal to the axis of rotation.
